(19) United States
(12) Patent Application Publication
(10) Pub. No.: US 2024/0081923 A1
(43) Pub. Date: Mar. 14, 2024

(54) CATHETERISATION ROBOT WITH A DRIVE COMPONENT SURROUNDED BY A SEALING RING

(71) Applicant: ROBOCATH, ROUEN (FR)

(72) Inventors: Marc BOULANGÉ, LA GAILLARDE (FR); Théo LEBEL, LILLE (FR)

(21) Appl. No.: 18/263,601

(22) PCT Filed: Feb. 7, 2022
(86) PCT No.: PCT/EP2022/052832

§ 371 Date: Jul. 31, 2023

(30) Foreign Application Priority Data
Feb. 8, 2021 (FR) FR2101184

(51) Int. Cl. A61B 34/30

(52) U.S. Cl. CPC A61B 34/30; A61B 2034/301

(57) ABSTRACT

Disclosed is a catheterization robot including: several tracks for longitudinal horizontal rectilinear translational movement of several flexible elongate medical elements, the tracks being parallel to each other; a drive component for driving a flexible elongate medical device in rectilinear translation, the drive component being common to the tracks, transversely horizontally movable, and surrounded by a sealing ring which is deformable and integrally secured to the drive component; a sealing belt which is transversely horizontally movable, is integrally secured to the periphery of the sealing ring, and of which two portions extend the sealing ring transversely horizontally, respectively on the two sides, sufficiently to ensure that all of the tracks remain continuously covered.

### FIELD OF THE INVENTION

The invention relates to the technical field of catheterization robots comprising one or more drive components for one or more flexible elongate medical elements, this or these drive components being respectively surrounded by one or more sealing rings. The flexible elongate medical elements are one or more catheters (for example stent or balloon type catheters) and/or one or more catheter guides.

### TECHNOLOGICAL BACKGROUND OF THE INVENTION

According to a first prior art, a catheterization robot is known in which each track for rectilinear translation has its own drive component for driving a flexible elongate medical element, which remains dedicated to this track for rectilinear translation.

According to a patent application for a catheterization robot (EP20217534, already filed by the Applicant but not yet published as of the filing date of the present patent application), a catheterization robot is described with several tracks for rectilinear translation of a flexible elongate medical element, and a drive component for a flexible elongate medical element that is common to said tracks, meaning either to several tracks of the catheterization robot or to all tracks of the catheterization robot, said drive component traveling from one track to another. Patent application EP20217534 is incorporated herein by reference.

When the drive component leaves a first track for a second track, it leaves this first track completely uncovered and therefore vulnerable to all kinds of pollution which could be deposited in this first track and thus quickly cause hygiene and sterility issues, or efficiency and operational issues over the long term.

It is conceivable to cover the entire catheterization robot with sterility cloths of complex shapes in order to properly isolate the track itself, which must be sterile, from the rest of the catheterization robot comprising the drive components and especially the actuators for these drive components which by default are often in a non-sterile motorization area.

However, this would present the disadvantage of a complex and impractical system, which would be particularly unsuitable for the numerous back-and-forth movements of the drive component(s) across the different tracks.

### OBJECTS OF THE INVENTION

The aim of the present invention is to provide a catheterization robot comprising at least one drive component for a flexible elongate medical element, said drive component being surrounded by a sealing ring, which at least partially overcomes the above disadvantages.

More particularly, the invention aims to provide a catheterization robot comprising at least one drive component for a flexible elongate medical element, said drive component being surrounded by a sealing ring, wherein the sterility at the translation tracks for the flexible elongate medical element per drive component is properly maintained, and this is achieved in a relatively simple and rather effective manner.

The invention therefore considers it advantageous to propose an effective system which is also simple, rapid, and practical, for protecting the track or tracks abruptly uncovered by their drive component when said drive component leaves them for another track.

To this end, the invention proposes a catheterization robot comprising: several tracks for longitudinal horizontal rectilinear translational movement of several flexible elongate medical elements, said tracks being parallel to each other; a drive component, for driving a flexible elongate medical element in rectilinear translation, which is common to said tracks and which is transversely horizontally movable so as to travel alternately from one track to another and which is surrounded by a sealing ring that is deformable so as to allow a relative, horizontal longitudinal displacement between said drive component and the periphery of said ring and that is integrally secured to said drive component so as to follow the transverse horizontal displacement of said drive component traveling from one track to another; a sealing belt which is transversely horizontally movable, which is integrally secured to said periphery of said sealing ring so as to follow the transverse horizontal displacement of said sealing ring and of said drive component traveling from one track to another, and of which two portions extend the sealing ring transversely horizontally respectively on the two sides, sufficiently so that the assembly formed by said sealing belt and by said sealing ring and by said drive component continuously covers all of said tracks, over the width of said sealing belt, during the travel of said drive component from one track to another.

The periphery of the sealing ring is the outer periphery of the sealing ring.

The drive components may be finger supports with their associated fingers, such as those described in patent application FR3066381, incorporated herein by reference (also in the name of the Applicant of this patent application).

According to some embodiments of the invention, it also concerns maintaining the seal between the sterile zone having the tracks(s) for movement of the flexible elongate medical element(s) on the one hand, and the interior of the articulated arm of the catheterization robot on the other hand, while allowing the movements of the drive components, for example the finger supports, from one flexible elongate medical element movement track to another flexible elongate medical element movement track.

According to some preferred embodiments, the invention comprises one or more of the following features which may be used separately or with some of them combined or with all of them combined.

Preferably, said sealing ring is deformable so as to also allow a relative vertical movement between said drive component and the periphery of said ring.

Thus, on the one hand, the change from one track to another track is facilitated for the drive component, even in the presence of a flexible elongate medical element within this track at the moment when the drive component leaves it, and on the other hand, the drive component has more space to perform one or more types of driving the flexible medical element other than a simple rectilinear translation, for example such as also rotating this flexible elongate medical element.

Preferably, the catheterization robot also comprises: another drive component for driving a flexible elongate medical element in rectilinear translation, said other drive component being common to said tracks and identical to said drive component, said two drive components being longitudinally offset relative to each other, and being synchronized with each other so as to alternate in driving a flexible elongate medical element in rectilinear translation, together forming a drive module for driving a flexible elongate medical element in rectilinear translation.

Thus, the advantageous system for protecting the track or tracks abruptly uncovered by their drive component when said drive component leaves them for another track, can easily be used for different types of catheterization robot, even complex ones, where a sterile cloth would be of increasingly complex shape.

Preferably, the catheterization robot comprises two drive modules, each comprising two drive components and each being common to said tracks.

The advantageous system for protecting track(s) abruptly uncovered by their drive component when said drive component leaves them for another track, can thus easily be used for different types of catheterization robots, even complex ones, where a sterile fabric would be of increasingly complex shape.

Preferably, the flexible elongate medical element is a catheter or a catheter guide.

Preferably, said tracks for longitudinal horizontal rectilinear translation of several flexible elongate medical elements, said tracks being parallel to each other, are four in number of which two tracks are for catheters and two tracks are for catheter guides.

The advantageous system for protecting track(s) abruptly uncovered by their drive component when said drive component leaves them for another track, can thus easily be used for different types of catheterization robots, even complex ones, where a sterile fabric would be of increasingly complex shape.

Preferably, said drive component comprises a pair of fingers arranged facing each other so as to grip or release between them a flexible elongate medical element, this pair of fingers being respectively carried by a pair of finger supports.

The drive components may be finger supports with their associated fingers, such as those described in patent application FR3066381, incorporated herein by reference (also in the name of the Applicant of this patent application).

Preferably, said sealing ring comprises a rigid frame at its periphery, and a deformable sealing bellows integrally secured around said drive component.

A seal is thus ensured for all tracks, not only for the tracks where the drive component is not or is no longer located, but also for the track where the drive component is located.

Preferably, said two portions of said sealing belt which extend said sealing ring, respectively terminate in two ends which join together to form a closed loop.

The sealing system is thus more robust with a closed loop than without a closed loop, because it reduces the risk of the sealing belt coming out of the grooves in which it is placed.

Preferably, said two portions of said sealing belt which extend said sealing ring respectively terminate in two free ends.

The sealing system is thus simpler without a closed loop than with a closed loop, because the sealing belt is less mechanically stressed and the structure of the sealing system is simpler overall.

Preferably, said tracks are carried by an intermediate portion of an articulated arm of the catheterization robot, and said two portions of said sealing belt which extend said sealing ring respectively terminate in two ends which each slide into a right-angled elbow with recess so as to limit the overall width, and preferably so as not to exceed the width of said intermediate portion of an articulated arm of the catheterization robot.

Thus, neither the presence of this sealing belt nor the path of movement of this sealing belt increases the size of the catheterization robot, since this path of movement of the sealing belt does not or barely extends beyond the width of said intermediate portion of an articulated arm of the catheterization robot carrying the various parallel tracks and defining the width of the catheterization robot.

Preferably, said two portions of said sealing belt which extend said sealing ring respectively terminate in two ends which each slide firstly into a right-angled elbow with recess so as to limit the overall width, and preferably so as not to exceed the width of said intermediate portion of an articulated arm of the catheterization robot, then into another right-angled elbow with recess so as to pass under said intermediate portion of an articulated arm of the catheterization robot.

Thus, neither the presence of this sealing belt nor the path of movement of this sealing belt increases the size of the catheterization robot, since this path of movement of the sealing belt does not or barely extends beyond the width of said intermediate portion of an articulated arm of the catheterization robot carrying the various parallel tracks and defining the width of the catheterization robot, even in the event of a long path of movement of this sealing belt corresponding in particular to a generally high number of tracks for rectilinear translation of a flexible elongate medical element, said tracks being arranged parallel to each other.

Preferably, said sealing belt is of a sufficiently reduced thickness compared to its width to be able to bend at a right angle and then return to being flat, without damage or wear.

The advantageous system for protecting track(s) abruptly uncovered by their drive component when said drive component leaves them for another track thus presents an improved compromise between robustness and size.

Preferably, said sealing belt is made of polymer.

The sealing belt is thus particularly flexible, including easily bending.

Preferably, said sealing belt is made of metal, preferably stainless steel.

The sealing belt is thus particularly robust and durable, while remaining sufficiently flexible due to a relatively low thickness chosen for the sealing belt.

Other features and advantages of the invention will become apparent upon reading the following description of a preferred embodiment of the invention, given as an example and with reference to the appended drawings.

### BRIEF DESCRIPTION OF DRAWINGS

FIG. 1 schematically represents a perspective view of an example of an articulated arm of a catheterization robot comprising at least one drive component for a flexible elongate medical element, said drive component being surrounded by a sealing ring, according to one embodiment of the invention.

FIG. 2 schematically represents a perspective view of an example of an intermediate portion of an articulated arm of a catheterization robot comprising at least one drive component for a flexible elongate medical element, said drive component being surrounded by a sealing ring, according to one embodiment of the invention.

FIG. 3 schematically represents a perspective view of an example of an assembly formed by a sealing belt and by a sealing ring on an intermediate portion of an articulated arm of a catheterization robot comprising at least one drive component for a flexible elongate medical element, said drive component being surrounded by a sealing ring, according to one embodiment of the invention.

FIG. 4 schematically represents a sectional view of an example of an assembly formed by a sealing belt and by a sealing ring on an intermediate portion of an articulated arm of a catheterization robot comprising at least one drive component for a flexible elongate medical element, surrounded by a sealing ring, according to one embodiment of the invention.

FIG. 5 schematically represents a sectional view of an example of an assembly formed by a sealing belt and by a sealing ring on an intermediate portion of an articulated arm of a catheterization robot comprising at least one drive component for a flexible elongate medical element, said drive component being surrounded by a sealing ring, according to another embodiment of the invention.

FIG. 6 schematically represents a perspective view of an example of a sealing belt sliding in a groove on an intermediate portion of an articulated arm of a catheterization robot comprising at least one drive component for a flexible elongate medical element, said drive component being surrounded by a sealing ring, according to one embodiment of the invention.

### DETAILED DESCRIPTION OF EMBODIMENTS OF THE INVENTION

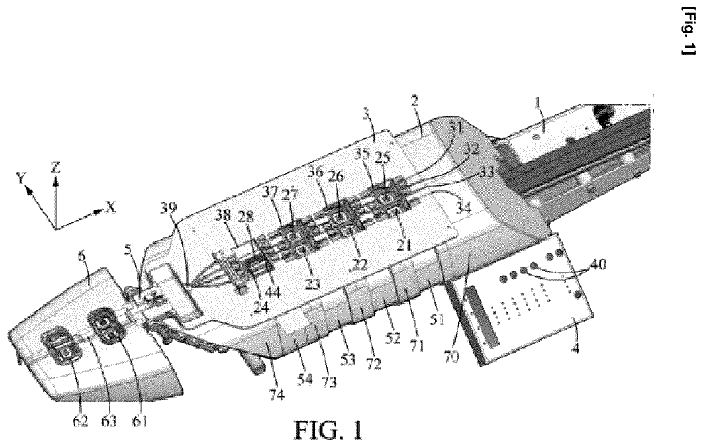

FIGS. 1 to 6 are identified within an orthogonal frame of reference XYZ, X being the longitudinal direction in the horizontal plane XY, Y being the transverse direction in the horizontal plane XY, Z being the vertical direction. The flexible elongate medical element is a catheter or a catheter guide.

FIG. 1 schematically represents a perspective view of an example of an articulated arm of a catheterization robot comprising at least one drive component for a flexible elongate medical element, said drive component being surrounded by a sealing ring, according to one embodiment of the invention.

The articulated arm of the catheterization robot successively comprises, from the user of the catheterization robot (the medical practitioner) towards the patient (who is being treated using this catheterization robot), from its most proximal part to its most distal part, a proximal portion 1 of the arm, an intermediate portion 2 of the arm, and a distal portion 6 of the arm. Attached to intermediate portion 2 of the arm is a panel 4 of controls 40. Between intermediate portion 2 of the arm and distal portion 6 of the arm is arranged a Y-connector type of connector (not shown in the figure), the Y connector being arranged within a fastening device 5. Distal portion 6 comprises a catheter guide (not shown in the figure) which can move in a rectilinear translational movement under the alternating and combined action of two drive components 61 and 62. The catheter guide can move in a guide track 63. Drive components 61 and 62 can be finger supports with their associated fingers, such as those described in patent application FR3066381, incorporated herein by reference. Arm portions 1, 2, and 6 are permanent elements of the catheterization robot.

Mounted on intermediate portion 2 of the catheterization robot arm, in a removable and detachable manner, is a plate 3 which is a single-use consumable and which is changed for each new patient.

Plate 3 comprises four entry tracks 31, 32, 33, 34 for rectilinear translation of the flexible elongate medical elements. These four entry tracks 31, 32, 33, 34 respectively have four openings oriented upwards through which four flexible elongate medical elements can be respectively inserted into these four entry tracks 31, 32, 33, 34 and can be extracted from these four entry tracks 31, 32, 33, 34. The ends of the rectilinear portion of these four entry tracks 31, 32, 33, 34 curve to converge towards each other in order to group together into a common exit track 39 before entering connector 5, which is a Y connector, i.e. a Y-shaped connector (connector with two inputs and one common output).

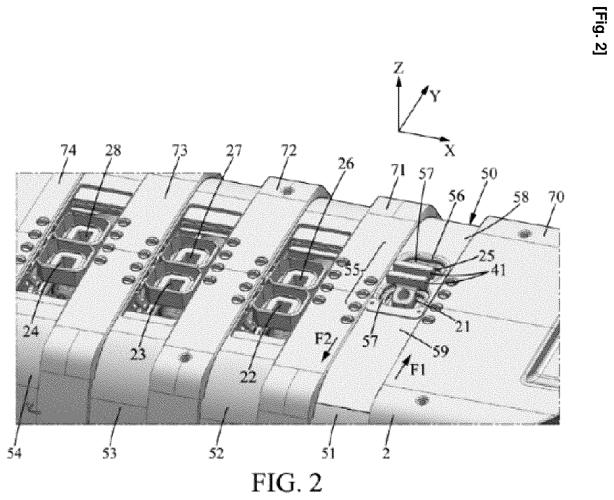

Plate 3 comprises four through-holes 35, 36, 37, 38, through which four drive components 41 to 44 (of which only drive component 44 is shown in FIG. 1, and of which only drive component 41 is shown in FIGS. 2, 5, and 6), divided into two contiguous pairs of drive components which alternately drive the flexible elongate medical elements in rectilinear translation respectively in the four tracks 31, 32, 33, 34. Drive components 41 to 44 can be finger supports with their associated fingers, such as those described in patent application FR3066381, incorporated herein by reference. The direction of rectilinear translation of the flexible elongate medical elements follows the common direction of the four entry tracks 31, 32, 33, 34, which are parallel to each other. The direction of rectilinear translation of the flexible elongate medical elements is the longitudinal direction X. The directional orientation of the rectilinear translation goes from proximal portion 1 of the arm towards distal portion 6 of the arm, conversely to the directional orientation of the X direction.

Through-holes 35, 36, 37, 38 respectively house pairs of corresponding housings 21 and 25, 22 and 26, 23 and 27, 24 and 28, in which drive components 41 to 44 are respectively mounted.

Intermediate portion 2 of the articulated arm of the catheterization robot comprises several transverse grooves (in the Y direction) 51, 52, 53, 54, which run respectively above the corresponding pairs of housings 21 and 25, 22 and 26, 23 and 27, 24 and 28.

Transverse groove 51 is located between transverse walls 70 and 71. Transverse groove 52 is located between transverse walls 71 and 72. Transverse groove 53 is located between transverse walls 72 and 73. Transverse groove 54 is located between transverse walls 73 and 74.

The rectilinear translation entry tracks 31, 32, 33, 34 are hollowed out of plate 3, and therefore form part of the single-use consumable part.

Entry track 31 receives and guides a first catheter which moves therein in rectilinear translation. Entry track 32 receives and guides a first catheter guide which moves therein in rectilinear translation. This first catheter guide is intended to be inserted into the first catheter at common exit track 39. Entry track 34 receives and guides a second catheter which moves therein in rectilinear translation. Entry track 33 receives and guides a second catheter guide which moves therein in rectilinear translation. This second catheter guide is intended to be inserted into the second catheter at common exit track 39.

FIG. 2 schematically represents a perspective view of an example of an intermediate portion of an articulated arm of a catheterization robot comprising at least one drive component for a flexible elongate medical element, said drive component being surrounded by a sealing ring, according to one embodiment of the invention.

Within transverse groove 51 slides a drive component 41 for rectilinear translation of a flexible elongate medical element, which is common to the various tracks 31, 32, 33, 34, along a transverse horizontal direction Y, so as to move alternately from one track to another. Drive component 41, for example a pair of finger supports respectively carrying a pair of fingers, is surrounded by a sealing ring 55.

Sealing ring 55 is deformable so as to allow on the one hand at least one relative movement, horizontal and longitudinal, along the X direction, between drive component 41 and periphery 56 of sealing ring 55, and advantageously on the other hand also a relative vertical displacement, along the Z direction, between drive component 41 and periphery 56 of sealing ring 55. Sealing ring 55 is integrally secured to drive component 41, and/or to housings 21 and/or 25 of drive component 41, so as to follow the transverse horizontal displacement, along the Y direction, of drive component 41 moving from one track to the other.

When drive component 41 goes from track 34 to track 31, it moves in rectilinear translation along the transverse horizontal Y direction, in the directional orientation of arrow F1, by sliding along groove 51. When drive component 41 goes from track 31 to track 34, it moves in rectilinear translation along the transverse horizontal Y direction, in the directional orientation of arrow F2, sliding along groove 51, the directional orientation of arrow F2 being opposite to the directional orientation of arrow F1. Within groove 51 also slides a sealing belt 50, along the transverse horizontal Y direction, following the movement of drive component 41 along the transverse horizontal Y direction.

Sealing belt 50 is integrally secured to periphery 56 of sealing ring 55 so as to follow, automatically and mechanically, the transverse horizontal displacement of sealing ring 55 along the Y direction, said ring itself being driven by the movement of drive component 41 traveling from one track to the other along groove 51. Sealing belt 50 has two portions 58 and 59 which extend sealing ring 55, in the transverse horizontal Y direction along groove 51, respectively on the two sides of sealing ring 55. The two portions 58 and 59 are long enough so that the assembly formed by sealing belt 50 and by sealing ring 55 and by drive component 41 continuously covers all tracks 31 to 34, over the width of sealing belt 50, during the travel of drive component 41 from one track to another, over its entire path of movement along the Y direction, all along groove 51.

It can be seen in FIG. 2 that the two portions 58 and 59 of sealing belt 50 which extend sealing ring 55 respectively terminate in two ends which each slide into a right-angled elbow with recess (see the curves of portions 58 and 59) so as to limit the overall width, and preferably so as not to exceed the width of intermediate portion 2 of the articulated arm of the catheterization robot. Sealing belt 50 is of a sufficiently reduced thickness compared to its width to be able to bend at a right angle and then return to being flat, without damage or wear. In one embodiment, the two portions 58 and 59 of sealing belt 50 which extend sealing ring 55 respectively terminate in two ends which meet to form a closed loop under intermediate portion 2 of the articulated arm of the catheterization robot, so as to extend all the way around intermediate portion 2 of the articulated arm of the catheterization robot. In another embodiment, the two portions 58 and 59 of sealing belt 50 which extend sealing ring 55 respectively terminate in two ends which remain free without meeting, and therefore without extending all the way around intermediate portion 2 of the articulated arm of the catheterization robot. In one embodiment, sealing belt 50 is made of polymer, thus making sealing belt 50 more flexible, while in another embodiment, sealing belt 50 is made of metal, preferably stainless steel, thus making sealing belt 50 stronger and more durable.

Sealing ring 55 comprises on the one hand a rigid frame at its periphery 56, its periphery 56 even being formed by rigid frame 56, and on the other hand a deformable sealing bellows 57 integrally secured around drive component 41 and/or around its housings 21 and/or 25. Rigid frame 56 surrounds sealing bellows 57 and in addition is integrally secured to sealing bellows 57. Advantageously, rigid frame 56 is integrally secured to drive component 41 and/or to its housings 21 and/or 25, so that a movement of drive component 41, in the transverse horizontal Y direction along groove 51, automatically and mechanically also drives the same movement of the sealing ring 55 and of the sealing belt 50 which is integrally secured thereto.

The catheterization robot advantageously comprises another drive component for driving a flexible elongate medical element in rectilinear translation (not shown in FIG. 2), which is common to tracks 31 to 34 and is identical to drive component 41, the two drive components then being longitudinally offset relative to one another, and synchronized with each other so as to alternate in driving a flexible elongate medical element (catheter or catheter guide) in rectilinear translation, thus together forming a drive module for driving a flexible elongate medical element in rectilinear translation. The catheterization robot even advantageously comprises two drive modules, each comprising two drive components and each being common to all tracks 31 to 34.

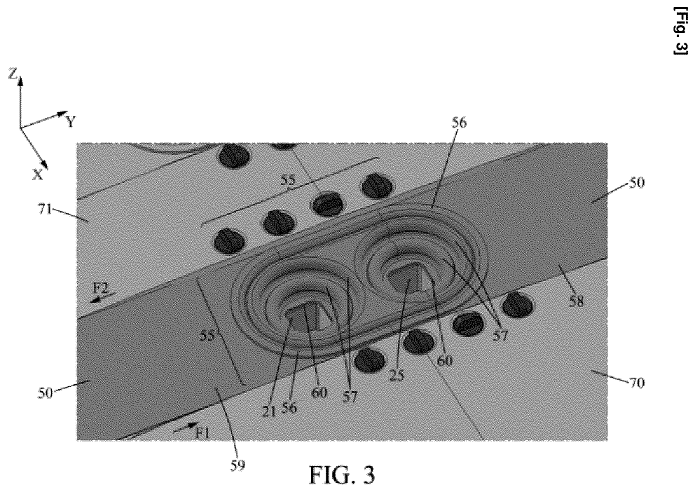

FIG. 3 schematically represents a perspective view of an example of an assembly formed by a sealing belt and by a sealing ring on an intermediate portion of an articulated arm of a catheterization robot comprising at least one drive component for a flexible elongate medical element, said drive component being surrounded by a sealing ring, according to one embodiment of the invention.

Sealing belt 50 is integrally secured on the two sides of sealing ring 55, by its two portions 58 and 59 which extend on each side of sealing ring 55. Sealing belt 50 has the same width along the longitudinal X direction than the width of sealing ring 55 along the longitudinal X direction.

Sealing ring 55 has the general shape of a flattened ellipse, i.e. formed of two circular ends interconnected by two rectilinear sections. Periphery 56, physically represented by rigid frame 56, also has the same general shape of a flattened ellipse, i.e. formed of two circular ends interconnected by two rectilinear sections.

Sealing bellows 57 has two square openings 60 under which can be seen housings 21 and 25 into which drive component 41 will be inserted. Between these two square openings 60 on the one hand and rigid frame 56 on the other hand, extend two pairs of concentric circular folds, advantageously two concentric circular folds.

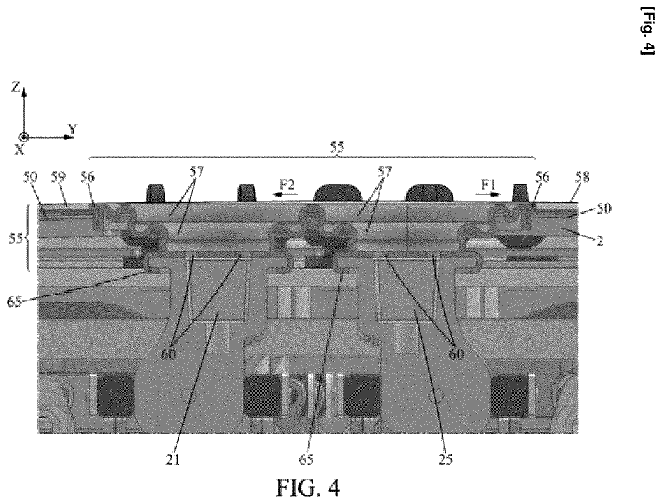

FIG. 4 schematically represents a sectional view of an example of an assembly formed by a sealing belt and by a sealing ring on an intermediate portion of an articulated arm of a catheterization robot comprising at least one drive component for a flexible elongate medical element, said drive component being surrounded by a sealing ring, according to one embodiment of the invention.

Sealing ring 55 shows in profile view its rigid frame 56 which has, over its entire circumference, a vertical body with a horizontal rim for better securing to sealing belt 50. The two concentric circular folds of sealing bellows 57 are located at different levels along the vertical Z direction. Under these two concentric circular folds of sealing bellows 57, in the vertical Z direction, sealing bellows 57 further comprises two hollowed-out hooks 65 respectively catching on two projections from the rest of intermediate portion 2 of the articulated arm of the catheterization robot.

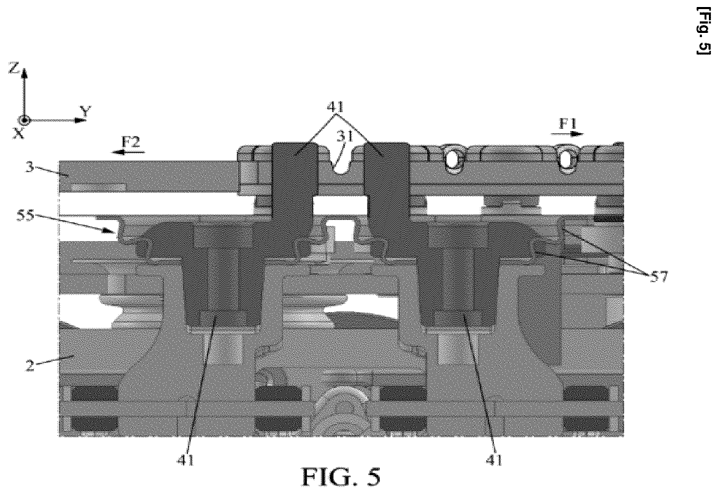

FIG. 5 schematically represents a sectional view of an example of an assembly formed by a sealing belt and by a sealing ring on an intermediate portion of an articulated arm of a catheterization robot comprising at least one drive component for a flexible elongate medical element, said drive component being surrounded by a sealing ring, according to another embodiment of the invention.

Sealing bellows 57 properly surrounds the two pins of the base of drive component 41. Sealing bellows 57 here constitutes the entirety of sealing ring 55 which then does not have a rigid frame.

Sealing belt 50 creates a sealing membrane which follows the movements of the pair of finger supports when drive component 41 changes track, closing off the rest of the opening made in intermediate portion 2 of the articulated arm of the catheterization robot all along groove 51, both in the directional orientation of arrow F1 and in the directional orientation of arrow F2.

Sealing bellows 57 also allows the downward and upward movements of the finger supports and fingers of drive component 41, along the vertical Z direction, in order to enable them to change tracks. In order to change tracks, the finger supports and fingers of drive component 41 descend and enter intermediate portion 2 of the catheterization robot, thus passing under the tracks 31 to 34 for the catheters and catheter guides, then place themselves in front of the catheter or catheter guide that is to be manipulated, then finally rise so that the pair of finger supports and fingers of drive component 41 can manipulate the desired catheter or guide.

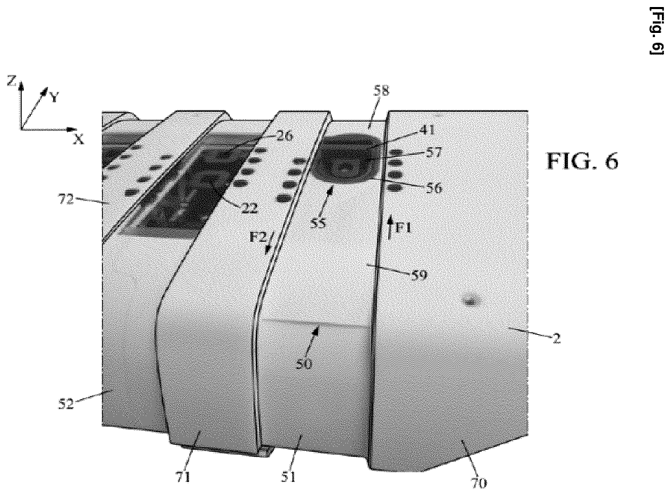

FIG. 6 schematically represents a perspective view of an example of a sealing belt sliding in a groove on an intermediate portion of an articulated arm of a catheterization robot comprising at least one drive component for a flexible elongate medical element, said drive component being surrounded by a sealing ring, according to one embodiment of the invention.

The bend in portion 59 of sealing belt 50 is clearly visible at the end of portion 59. Sealing belt 50 is properly guided in groove 51, by transverse walls 70 and 71, when it slides along the transverse horizontal Y direction. Projecting from the two transverse walls 70 and 71, two horizontal projections cover sealing belt 50 in the vicinity of transverse walls 70 and 71, in order to hold sealing belt 50 in place in groove 51, limiting its degree of freedom along the vertical Z direction.

Sealing belt 50 can have a thickness of between 0.05 mm and 0.5 mm Sealing belt 50 can in particular have a thickness of between 0.05 mm and 0.2 mm when said sealing belt 50 is made of metal (in particular of stainless steel), and preferably a thickness of between 0.07 mm and 0.15 mm Sealing belt 50 may in particular have a thickness of between 0.1 mm and 0.5 mm when said sealing belt 50 is made of polymer, and preferably a thickness of between 0.2 mm and 0.4 mm. The thickness of sealing belt 50 can be variable over the length of said sealing belt 50, the thickness of the belt possibly being lower on the portions of said sealing belt 50 which are capable of bending in order to follow the curvatures of the casing of the catheterization robot.

Of course, the invention is not limited to the examples and to the embodiment described and represented, but is capable of numerous variants accessible to those skilled in the art. In particular, the number of tracks and of drive components may vary in comparison to the number of variants shown in the figures.

1. Catheterization robot comprising:
several tracks for longitudinal horizontal rectilinear translational movement of several flexible elongate medical elements, said tracks being parallel to each other,
a drive component for driving a flexible elongate medical element in rectilinear translation, which is:
common to said tracks,
transversely horizontally movable so as to travel alternately from one track to another,
surrounded by a sealing ring which is:
deformable so as to allow a relative, horizontal longitudinal displacement between said drive component and the periphery of said ring,
integrally secured to said drive component so as to follow the transverse horizontal displacement of said drive component traveling from one track to another,
a sealing belt:
which is transversely horizontally movable,
which is integrally secured to said periphery of said sealing ring so as to follow the transverse horizontal displacement of said sealing ring and of said drive component traveling from one track to another,
and of which two portions extend the sealing ring:
transversely horizontally, respectively on the two sides,
sufficiently so that the assembly formed by said sealing belt and by said sealing ring and by said drive component continuously covers all of said tracks, over the width of said sealing belt, during the travel of said drive component from one track to another.

2. The catheterization robot according to claim 1, wherein said sealing ring is deformable so as to also allow a relative vertical movement between said drive component and the periphery of said ring.

3. The catheterization robot according to claim 1, further comprising:
another drive component for driving a flexible elongate medical element in rectilinear translation, said other drive component being:
common to said tracks,
identical to said drive component, said two drive components being:
longitudinally offset relative to each other,
synchronized with each other so as to alternate in driving a flexible elongate medical element in rectilinear translation, together forming a drive module for driving a flexible elongate medical element in rectilinear translation.

4. The catheterization robot according to claim 3, comprising two of the drive modules, each comprising two drive components and each being common to said tracks.

5. The catheterization robot according to claim 1, wherein the flexible elongate medical element is a catheter or a catheter guide.

6. The catheterization robot according to claim 1, wherein said tracks for longitudinal horizontal rectilinear translation of several flexible elongate medical elements, said tracks being parallel to each other, are four in number of which two tracks are for catheters and two tracks are for catheter guides.

7. The catheterization robot according to claim 1, wherein said drive component comprises a pair of fingers arranged facing each other so as to grip or release between them a flexible elongate medical element, this pair of fingers being respectively carried by a pair of finger supports.

8. The catheterization robot according to claim 1, wherein:
said sealing ring comprises:
a rigid frame at its the periphery of the sealing ring,
a deformable sealing bellows integrally secured around said drive component.

9. The catheterization robot according to claim 1, wherein said two portions of said sealing belt which extend said sealing ring respectively terminate in two ends which join together to form a closed loop.

10. The catheterization robot according to claim 1, wherein said two portions of said sealing belt which extend said sealing ring respectively terminate in two free ends.

11. The catheterization robot according to claim 1, wherein:
said tracks are carried by an intermediate portion of an articulated arm of the catheterization robot,
said two portions of said sealing belt which extend said sealing ring respectively terminate in two ends which each slide into a right-angled elbow with recess so as to limit the overall width.

12. The catheterization robot according to claim 11, wherein said two portions of said sealing belt which extend said sealing ring respectively terminate in two ends which each slide firstly into a right-angled elbow with recess so as to limit the overall width, then into another right-angled elbow with recess so as to pass under said intermediate portion of an articulated arm of the catheterization robot.

13. The catheterization robot according to claim 1, wherein said sealing belt is of sufficiently reduced thickness compared to the sealing belt's its width to be able to bend at a right angle and then return to being flat, without damage or wear.

14. The catheterization robot according to claim 1, wherein said sealing belt is made of polymer.

15. The catheterization robot according to claim 1, wherein said sealing belt is made of metal.

16. The catheterization robot of 11, wherein said two portions of said sealing belt which extend said sealing ring respectively terminate in two ends which each slide into a right-angled elbow with recess so as not to exceed the width of said intermediate portion of an articulated arm of the catheterization robot.

17. The catheterization robot of 12, wherein said two portions of said sealing belt which extend said sealing ring respectively terminate in two ends which each slide firstly into a right-angled elbow with recess so as not to exceed the width of said intermediate portion of an articulated arm of the catheterization robot.

18. The catheterization robot of claim 15, wherein the sealing belt comprises stainless steel.
